(19) United States
(12) Patent Application Publication
(10) Pub. No.: US 2010/0130835 A1
(43) Pub. Date: May 27, 2010

(54) METHODS FOR SCREENING AND TREATING PATIENTS WITH COMPROMISED CARDIOPULMONARY FUNCTION

(75) Inventors: Rodney Brenneman, San Juan Capistrano, CA (US); Brad Kellerman, Escondido, CA (US); J. Christopher Flaherty, Topsfield, MA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Rox Medical, Inc., San Clemente, CA (US)

(21) Appl. No.: 12/570,506

(22) Filed: Sep. 30, 2009

Related U.S. Application Data
(60) Provisional application No. 61/101,612, filed on Sep. 30, 2008.

(51) Int. Cl. A61B 5/02; A61B 5/00; A61B 17/00

(52) U.S. Cl. 600/301; 600/481; 128/898

(57) ABSTRACT

Methods are described for screening patients suffering from compromised cardiopulmonary function to determine if they will benefit from arteriovenous fistula therapy. Performance of an exercise regimen by the patients both in the presence and absence of supplemental oxygen is measured. Those patients whose performance improves in the presence of supplemental oxygen are considered likely candidates for benefitting from arteriovenous therapy.

### CROSS REFERENCE TO RELATED APPLICATIONS

This application claims the benefit of prior provisional application No. 61/101,612 (Attorney Docket No. 022102-000800US), filed on Sep. 30, 2008, the full disclosure of which is incorporated herein by reference.

### BACKGROUND OF THE INVENTION

1. Field of the Invention

The present invention relates to methods for screening patients to determine whether they will respond to a medical treatment. More particularly, the present invention relates to methods for screening patients suffering from compromised cardiopulmonary function to determine whether they will respond to treatment involving the formation of an arteriovenous fistula.

Cardiopulmonary function can be affected by a variety of common disorders, including chronic obstructive pulmonary disease (COPD), certain cardiac diseases, systemic hypertension and hypotension, emphysema, and other lung conditions. Many if not all of these conditions can result in insufficient blood oxygenation, causing the patient to tire easily and have difficulty breathing.

Recently, arteriovenous fistula therapy (AVF) has been proposed as a method for increasing blood oxygenation and treating patients suffering from compromised cardiopulmonary function. Arteriovenous fistula therapy is performed by creating a fistula between the arterial circulation and the venous circulation in order to raise the mixed venous oxygen content and lessen the adverse consequences of venous admixture in patients with chronic lung disease. While AVF therapy has been successful in many patients suffering from compromised cardiopulmonary function, the success is not uniform among all such patients.

For these reasons, it would be desirable to be able to screen and identify those patients suffering from compromised cardiopulmonary function who are more likely to respond to AVF therapy than those patients who are less likely to respond. Desirably, such screening methods would be non-invasive and present minimum risk to the patients being tested. At least some of these objectives will be met by the inventions described hereinafter.

2. Description of the Background Art

Methods and devices for performing arteriovenous fistula therapy (AVF), are described in the following co-pending applications, each of which is incorporated in its entirety herein by reference: Ser. No. 10/820,169; Ser. No. 11/961,731; Ser. No. 11/152,284; Ser. No. 11/013,981; Ser. No. 11/152,621; Ser. No. 11/151,802; Ser. No. 11/282,341; Ser. No. 11/356,876; Ser. No. 11/696,635; Ser. No. 11/946,454; and Ser. No. 12/017,437.

The effects of supplemental oxygen delivered during exercise to patients suffering from chronic obstructive pulmonary disease are described in Jolly et al. (2001), Chest 120:437-443. The data presented in the Experimental section of the present application was first presented at the American Thoracic Society Meeting, in Chicago, Ill., on Oct. 23, 2007.

### BRIEF SUMMARY OF THE INVENTION

The present invention provides methods for screening patients suffering from compromised cardiopulmonary function to determine if those patients are likely to benefit from arteriovenous fistula therapy (AVF). The methods rely on determining (or providing data representative of) a patient's ability to perform an exercise regimen in both the presence and absence of supplemental oxygen. By comparing the patient's ability to perform the exercise regimen in the presence and absence of oxygen, it can be determined whether the patient will likely benefit from AVF therapy. In particular, those patients who are able to improve their performance of the exercise regimen in the presence of supplemental oxygen will likely benefit from receiving the AVF therapy while those who show little or no improvement in the presence of supplemental oxygen will be less likely to benefit from the AVF therapy.

The screening methods of the present invention will be particularly suitable for patients suffering from chronic obstructive pulmonary disease (COPD), but will also be suitable for screening patients suffering from other cardiopulmonary conditions, such as congestive or other heart failure, hypertension, hypotension, coronary artery disease, respiratory failure, lung fibrosis, adult respiratory distress syndrome (ARDS), chronic bronchitis, emphysema, cystic fibrosis, cystic lung disease, chronic asthma, and the like. The screening method of the present invention is typically performed after the performance of a diagnostic procedure whose results indicate compromised cardiopulmonary function of the patient, such as reduced ability to exercise, hypoxemia, dypsnea, and the like.

The exercise regimen will typically involve aerobic exercise, i.e. exercise where the patient uses large muscle groups and places demands on the cardiovascular system and where performance depends on the patient's efficiency of oxygen use. While a variety of aerobic exercises exist, such as walking, jogging, running, swimming, bicycling, rowing, and the like, the screening methods of the present invention will preferably rely on exercise regimens which do not place too great a burden on the patient, as most if not all the patients undergoing this testing will have compromised lung function and be unable to perform vigorous exercises. Thus, it has been found that walking provides a very useful form of exercise where performance can be measured both in the presence and absence of oxygen. The walking can be done on a fixed walking surface, such as a measured track or hallway, but can also be done on a treadmill which simplifies the need to transport oxygen. The present invention, however, is not limited to walking as the exercise regimen and any of the other aerobic exercises listed above could find use in alternative protocols.

While the exercise regimens performed with and without the patient receiving supplemental oxygen will usually be the same or identical, the regimen performed in the presence of supplemental oxygen may differ from that performed without supplemental oxygen, usually being more rigorous, making the patient more sensitive to the benefit of the supplied oxygen. The exercise regimen performed without supplemental oxygen will usually (but not necessarily) be performed prior to that with supplemental oxygen to reduce the likelihood of false positives which could occur if the patient is more tired during the second test.

When walking is the exercise regimen, the supplemental oxygen can be provided using an ambulatory oxygen source, such as an oxygen bottle with a nasal oxygen feed line. The patient can wear the supplemental oxygen feed while walking with the bottle, or the oxygen can be stationary if the patient is on a treadmill, stepper, rowing machine, exercise bicycle, elliptical trainer, or similar non-ambulatory exercise device. The supplemental oxygen feed may be attached to or carried by the patient during testing without supplemental oxygen delivery, in order to provide a control or “placebo test” where the patient, the technician and/or clinician performing the test is unaware of which exercise regimen included the delivery of oxygen. Test apparatus may be configured to deliver room air or oxygen, based on one or more controls blinded to the patient and/or performer of the test.

Walking performance can be measured based on the distance the patient can cover in a particular time period, or the amount of time it takes the patient to walk a predetermined distance. In the Experimental section below, patients were allowed to walk for six minutes both in the presence and absence of oxygen, and the distance walked was measured. The time that the patient walks, however, could be varied to be shorter or longer. Alternatively or additionally, other patient performance and/or physiologic parameters can be measured during one or more of the exercise regimens (e.g. during a first exercise regimen without supplemental oxygen and during a second exercise regimen with supplemental oxygen), and compared to determine if the patient is a candidate for arteriovenous fistula therapy. Such patient parameters are indicative of cardiopulmonary function and include but are not limited to: heart rate; respiration rate; blood oxygen, desaturation or desaturation rate; dyspnea; oxygen consumption; oxygen delivery; pulmonary vascular resistance; cardiac output; and combinations of these.

All other conditions of the patient should be controlled carefully so that the only variable experienced by the patient is whether or not supplemental oxygen is present. In particular, the patient should not take drugs, eat, or perform any other activities between the times where the exercise performance is measured. Typically, the exercise performance will be measured on the same day, usually with a short rest between the performance with oxygen and without oxygen typically less than 24 hours, but greater than 30 minutes. The order in which the regimen is performed is not critical and it is necessary only that the patient be in substantially the same condition at the beginning of each exercise regimen, typically being in a resting condition with a base level heart rate. In some cases, it may be desirable to measure the patient's heart rate to assure that it has returned to a base level before beginning at least the second (comparison) exercise regimen, often before both exercise regimens. The patient may be restricted from taking one or more drugs, such as a COPD therapy drug, for a time period prior to performing an exercise regimen, such as a time period of multiple hours to multiple days. Numerous other patient parameters may be controlled prior to and/or during performance of the exercise regimen. Typical patient parameters include but are not limited to: flow rate of oxygen during exercise regimens; drug dose prior to exercise regimens; caloric intake prior to, during, and/or between exercise regimens; patient activity prior to exercise regimens; duration of time between first and second exercise regimen; patient exertion during exercise regimens; and combinations of these.

Those patients who are likely candidates for benefitting from the AVF therapy will be those whose performance of the exercise regimen improves in the presence of oxygen in comparison with their performance in the absence of supplemental oxygen. In the case of walking, improvements predictive of the benefits of AVF therapy will typically be at least about 5% in the measured distance for a relatively short walk, e.g. below 15 minutes, usually below 10 minutes, and often at 6 minutes as shown in the Experimental section hereinafter. It will also be possible to provide a qualitative determination of how likely an individual patient is to benefit from the AVF therapy depending on the degree of improvement in performance. That is, those patients who improve in the walking test by 10% will be more likely to benefit from the AVF therapy than those who improve by only 5%. Similarly, those who improve by 15% in the walking test will be more likely to improve, or may expect a greater improvement, than those who improve less in the supplemental oxygen test.

The present invention further provides methods for treating patients to improve cardiopulmonary function. The method comprises identifying patients as candidates for arteriovenous fistula (AVF) by any of the methods described above. Those patients who are identified as candidates are then treated by forming an arteriovenous fistula to enhance oxygen delivery to the patient. The AVF therapy will be performed as described in any of the related, co-pending applications listed and incorporated by reference above. In the exemplary embodiments, the AVF therapy is performed by creating an anastomosis between an artery and a vein distal to the renal arteries and veins. A broad range of arteries and veins can be chosen for fistula locations including but not limited to: common or external iliac artery and vein, femoral artery, saphenous vein, axillary artery and vein, subclavian artery and vein, axillary artery and vein; brachial artery and vein; poplitieal artery and vein, ulner artery; radial artery; profundal artery; basilica vein, cephalic vein, medial forearm vein, medial cubital vein, the aorta, and the inferior vena cava. The anastomosis is typically a side-to-side anastomosis, and such side-to-side anastomoses will preferably be performed by endovascular placement of an anastomotic connector. Alternatively, an end-to-side anastomosis may be created, such as by connecting a graft between an artery and a vein (two end-to-side anastomosis), or by anastomosing the severed end of an artery (e.g., a single anastomosis with the Left Internal Mammary Artery or Right Internal Mammary Artery) to a vein. The resultant flow rate of the fistula is targeted to be less than 1.5 liters/minute, preferably between 0.8 to 1.0 liters per minute.

The screening methods of the present invention are useful to determine if patients who previously had a procedure to create a first therapeutic fistula might benefit from a further procedure to enhance the AVF therapy. Such enhancement may be accomplished by simply modifying (usually enlarging) the existing fistula to increase the flow rate, e.g., by increasing the cross sectional area of the fistula via balloon dilation. Prior to enlarging the fistula, a fistula flow rate measurement may be performed in order to confirm the inadequacy of the existing fistula flow rate. Alternatively, a second fistula may be created in addition to existing fistula.

### DETAILED DESCRIPTION OF THE INVENTION

Experimental. METHODS: Twelve patients (10 men, 2 women) with severe COPD were selected. Mean (SD) age was 66(6) years, postbronchodilator FEV1 21 (8) %. DLCO 39 (14) %. Baseline PaO₂ was 58 (3) mmHg and PaCO₂ 44 (5) mmHg breathing room air (RA). Exercise performance was assessed by 6-minute walking distance (dw6), breathing RA then supplemental oxygen (O₂), at baseline, 6 weeks and 12 weeks after creating an AVF. Fistulas were created surgically or percutaneously in the iliofemoral region. Their luminal diameters ranged from 3 to 5 mm.

RESULTS: At baseline, O₂ increased mean dw6 by 62 m (P=0.02). In 5 subjects (responders) there was a clinically meaningful increase >54 in. After creating the AVF, mean dw6 for all patients (breathing RA) increased by 56 m at 6 weeks (P=0.04) and by 59 m (P=0.02) at 12 weeks. Responders increased dw6 by 129 m at 6 weeks (P=0.02) and 124 m at 12 weeks (P<0.01). Non-responders showed insignificant changes in dw6 after 6 weeks (3 m) and 12 weeks (13 m). An exercise response to O₂ at baseline was clearly associated with an exercise response to the fistula (Fisher's Exact P=0.02).

CONCLUSION: Therapeutic creation of an AVF increased exercise performance in COPD, presumably by raising mixed venous oxygen content and lessening the adverse consequences of venous admixture. Improved exercise performance breathing supplemental O₂ predicted which patients obtained this unique benefit.

CLINICAL IMPLICATIONS: This study reveals that exercise performance in severe COPD can be improved by a simple procedure. Furthermore, patients likely to benefit may be selected by their response to supplemental O₂ and a similar improvement in functional exercise capacity may be anticipated with a fistula.

While the above is a complete description of the preferred embodiments of the invention, various alternatives, modifications, and equivalents may be used. Therefore, the above description should not be taken as limiting the scope of the invention which is defined by the appended claims.

What is claimed is:

1. A method for screening patients prior to arteriovenous fistula therapy (AFT) to improve cardiopulmonary function, said method comprising:
determining a patient's ability to perform a first exercise regimen in the absence of supplemental oxygen;
determining the patient's ability to perform a second exercise regimen while receiving supplemental oxygen;
comparing the patient's ability to perform the first exercise regimen with the patient's ability to perform the second exercise regimen;
identifying the patient as a candidate for arteriovenous fistula therapy if the ability to perform the second exercise regimen improves relative to the ability to perform the first exercise regimen.

2. A method as in claim 1, wherein identifying comprises identifying those patients whose performance of the second exercise regimen in the presence of supplemental oxygen improve, by at least 5%.

3. A method as in claim 2, wherein the performance improves by at least 10%.

4. A method as in claim 2, wherein the performance improves by at least 15%.

5. A method as in claim 1, wherein determining the patient's ability to perform the exercise regimens comprises measuring the distance the patient can walk over a predetermined time period or measuring the time it takes the patient to walk a fixed distance.

6. A method as in claim 5, wherein the distance increases or time decreases by at least 5% in order for the patient to be identified as a candidate for arteriovenous fistula treatment.

7. A method as in claim 5, wherein the distance increases or time decreases by at least 10% in order for the patient to be identified as a candidate for arteriovenous fistula treatment.

8. A method as in claim 5, wherein the distance increases or time decreases by at least 15% in order for the patient to be identified as a candidate for arteriovenous fistula treatment.

9. A method as in claim 1, wherein the first exercise regimen is the same as the second exercise regimen.

10. A method as in claim 1, wherein the second exercise regimen is more rigorous than the first exercise regimen.

11. A method as in claim 1, wherein the first exercise regimen is performed before the second exercise regimen.

12. A method as in claim 1, wherein the second exercise regimen is performed before the first exercise regimen.

13. A method as in claim 1, further comprising performing a cardiopulmonary diagnostic procedure prior to determining if the patient is a candidate for the AFT screening.

14. A method as in claim 1, wherein the patient refrains from taking drugs which promote cardiopulmonary function prior to AFT screening.

15. A method as in claim 1, further comprising controlling a patient parameter selected from the group consisting of oxygen intake, drug administration, caloric intake, and prior physical activity.

16. A method as in claim 1, wherein the time between the first and second exercise regimens is in the range between 30 minutes and 24 hours.

17. A method as in claim 1, wherein the patient is blinded as to when they are receiving supplemental oxygen.

18. A method as in claim 1, wherein a tester administering the method to the patient is blinded as to when the patient is receiving supplemental oxygen.

19. A method as in claim 1, wherein determining the patient's ability to perform the exercise regimens comprises measuring heart rate, respiration rate, blood oxygen desaturation or desaturation rate, dyspnea, oxygen consumption, oxygen delivering, pulmonary vascular resistance, and cardiac output.

20. A method for treating patients to improve cardiopulmonary function, said method comprising:
identifying patients as candidates for arteriovenous fistula therapy as set forth in claim 1; and
forming or modifying an arteriovenous fistula in those patients identified as candidates.

21. A method as in claim 20, wherein forming an arteriovenous fistula comprises creating an anastomosis between an aorta and an inferior vena cava distal to the renal arteries and veins.

22. A method as in claim 21, wherein forming comprises creating a side-to-side or end-to-side anastomosis.

23. A method as in claim 22, wherein creating the side-to-side anastomosis comprises the endovascular placement of an anastomotic connector.

24. A method as in claim 20, wherein modifying an arteriovenous fisture comprises increasing the blood flow rate through the fistula.

25. A method as in claim 20, wherein a second arteriovenous fistula is formed.

26. A method as in claim 21, further comprising measuring flow through an existing arteriovenous fistula before and after it is modified.
